(19) United States
(12) Patent Application Publication
(10) Pub. No.: US 2010/0063439 A1
(43) Pub. Date: Mar. 11, 2010

(54) INJECTION DEVICE

(75) Inventor: Peter Eichhorst, Hohen Neuendorf (DE)

Correspondence Address: Nixon Peabody LLP, 200 Page Mill Road, Suite 200, Palo Alto, CA, 94306, US

(73) Assignee: PRIMOJEX GMBH, Hohen Neuendorf (DE)

(21) Appl. No.: 12/376,572

(22) PCT Filed: Aug. 23, 2007
(86) PCT No.: PCT/EP2007/058784

§ 371 Date: Feb. 26, 2009

(30) Foreign Application Priority Data
Aug. 31, 2006 (DE) 10 2006 041 499.3

(51) Int. Cl. A61M 5/30; A61M 5/31

(52) U.S. Cl. 604/68; 604/414

(57) ABSTRACT

The invention relates to an injection device, especially for a device for injecting an injectable product. Said device comprises a reservoir (12) for receiving the product, a first outlet arrangement (14) comprising a needle and used to transfer the product from the reservoir (12) to the injection site, and an actuating device (16) for applying a pressure (P) to the product in the reservoir (12), the actuating device (16) comprising a second, needleless outlet arrangement (44, 54) for transferring the product from the reservoir (12) to the injection site or being arranged against the second needleless outlet arrangement (46, 56) in a replaceable manner.

The invention relates to an injection device, in particular for a system for injection of an injectable product.

WO 03/105934 A1 describes a device for needle-free injection of a medium into human or animal tissue as well as a device for needle-free creation of an injection channel in human or animal tissue for guiding a medium to be injected into the tissue. This provides for a preinjection device for creating a high-pressure stream for creating a preinjection medium for creating an injection channel by means of a high pressure and small volume and a main injection device for introducing the medium to be injected with a large volume and a low pressure in comparison with the volume and pressure of the preinjection device. The preinjection device and the main injection device may each have separate chambers for the medium to be injected or one combined chamber.

DE 103 40 613 A1 also describes a system for injection of an injectable product, with a supply container that holds the product, an outlet arrangement for transferring the product from the supply container and an operating device for applying a pressure to the product in the supply container, such that the device may optionally be used as a disposable syringe or as an ampoule for a needle-free injector.

These known devices are used for injection of an injectable product, where an injectable product is understood to refer in particular to liquids, e.g., solutions, suspensions or dispersions containing an active ingredient. Active ingredients may be active pharmaceutical substances for treating the human or animal body or substances for diagnostic or cosmetic use. Furthermore, the injectable products may also serve to supply active ingredients to plants.

The known devices here are used, for example, for subcutaneous, intravenous, intramuscular or intracutaneous administration of the injectable products. The administration of the injectable product is accomplished by an injection using a cannula, for example. So-called disposable syringes are known for use here, comprising a supply container that can be filled with the product to be injected, which is connected to a cannula covered by a safety cap and for which, on the other hand, an operating device is also provided, by means of which a plunger is displaceable inside the supply container. For use of this disposable syringe, it is removed from a sterile blister package and the safety cap is removed from the cannula; then, after placing the disposable syringe in the region of tissue of the body where the injection is to be administered, the product to be injected is transferred into the tissue by pressing on the operating device.

On the other hand, there are known so-called needle-free injectors, by means of which a product to be injected is applied under pressure to an area of tissue, so that it enters the tissue under pressure. Such needle-free injectors have a holding area for a prefilled ampoule containing the injectable product, an operating device for applying a pressure to the product, so that the product emerges through a nozzle arrangement and penetrates into the desired tissue areas. The application of pressure may be accomplished here by a release element that is prestressable against the force of a spring element or by an external pressure medium.

Whether a disposable syringe or a needle-free injector is used will depend on the user group for the product to be injected, among other things.

The object of the present invention is to create an injection device, in particular for a system for injection of an injectable product, which is characterized by a simple design and will allow flexible use in administration of injectable products.

According to the invention, this object is achieved by an injection device having the features defined in Claim 1. Due to the fact that the injection device comprises a supply container holding the product to be injected, a first outlet arrangement equipped with a needle for transferring the product out of the supply container to the injection site, an operating device for applying pressure to the product in the supply container, such that the operating device has a second outlet arrangement which is needle-free for transferring the product out of the supply container to the injection site or is arranged to be replaceable by the second needle-free outlet arrangement, it is advantageously possible to use the injection device as a conventional prefilled standard disposable needle syringe on the one hand or to use this disposable syringe as a disposable product for needle-free injection. In this way, it is advantageously possible to decide, in particular just before administering the product to be injected, whether it is to be administered conventionally as a manual needle injection using the disposable syringe or as a needle-free injection. This therefore yields the possibility of using the same primary packaging means for the injectable product optionally as a disposable syringe or as a disposable product for needle-free injection.

In a preferred embodiment of the invention, the needle-free outlet arrangement has a plunger comprising a nozzle arrangement (needle-free injection) and a fluid connection between the supply container of the injection device and the nozzle arrangement, such that the nozzle arrangement preferably comprises an interior space that is connected to the fluid connection (hollow needle) via at least one outlet opening. Such an embodiment advantageously achieves the result that the standard syringe plunger can be replaced by a double-function plunger (NFI plunger) for the purpose of retrograde barrel support for the dispersion phase of the injection, such that the NFI plunger functionally simulates the penetration phase of the injection. By introducing a needle-free injector that can be used one or more times, conversion of the injection device is readily possible. The NFI plunger is initially joined to the stopper remaining in the syringe barrel. The stopper is punctured only after the NFI plunger is joined to the syringe barrel and inserted into the injection device. Furthermore, it is advantageously possible for an initial transfer of a small portion of the product to be injected from the syringe barrel into the NFI plunger to take place automatically when the injection device with the modified injection system is placed tightly on the injection site (skin). Triggering of the injection, which is controlled by contact pressure, is initiated by release of the dispersion force (retrograde barrel support with a stationary NFI plunger) and delayed activation of the penetration pulse in the NFI plunger while at the same time maintaining the dispersion force. The dispersion force is of a size such that it is within the range of the maximum thumb pressure force in conventional syringe operation. The return-inhibiting properties of the NFI plunger suppress a retrograde pressure pulse effect on the syringe barrel and therefore prevent rupture of the syringe barrel.

Additional preferred embodiments of the invention are derived from the other features defined in the dependent claims.

The invention will be explained in greater detail below in an exemplary embodiment on the basis of the respective figures, in which:

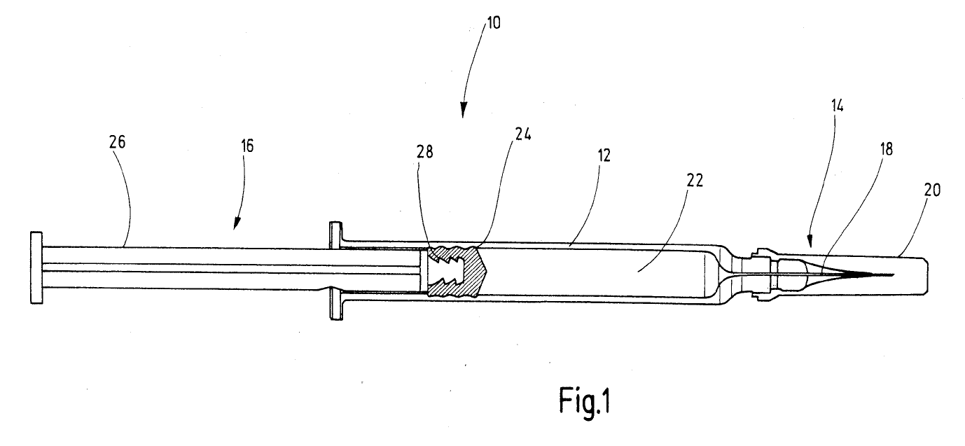

FIG. 1 shows schematically a sectional diagram through a disposable syringe;

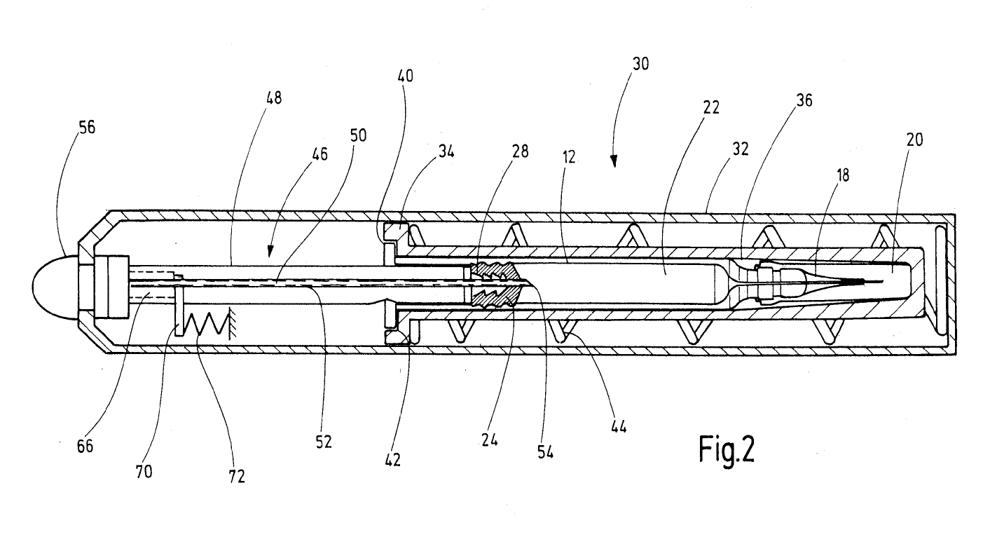

FIG. 2 shows schematically a sectional diagram through a device for needle-free injection;

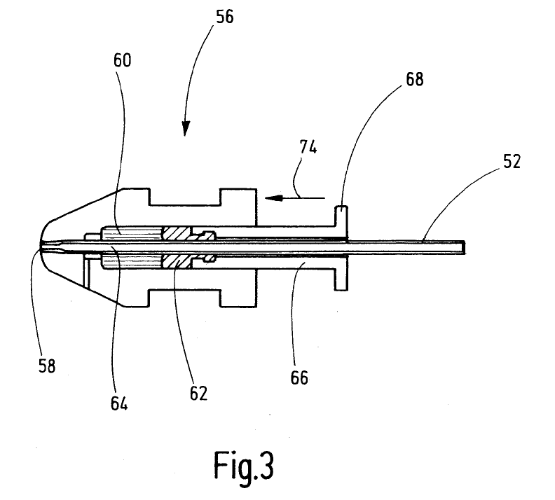

FIG. 3 shows schematically a sectional view through a nozzle arrangement of the needle-free injector; and

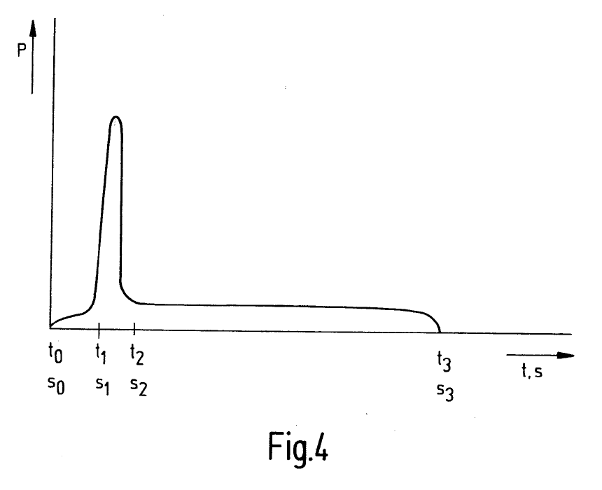

FIG. 4 shows a formula diagram.

FIG. 1 shows an injection device labeled as 10 on the whole for injection of an injectable product. The injection device 10 comprises a supply container 12, an outlet arrangement, which is referred to subsequently as the first needle-equipped outlet arrangement 14, and an operating device 16.

The first outlet arrangement 14 comprises a cannula 18, which is integrated into the supply container 12. The cannula 18 is covered by a safety cap 20. The safety cap 20 is attachable, clippable or the like onto the supply container 12 by means of suitable shape features.

The supply container 12 is formed by a cylindrical hollow body having an interior space 22. The hollow body is made of glass, for example, in particular borosilicate glass, but it may also be made of another suitable material, for example, plastic, metal or the like.

The interior space 22 is bordered by a stopper 24, which communicates with a plunger rod 26. The stopper 24 is made of butyl rubber, for example, while the plunger rod 26 is made of plastic, for example. The plunger rod 26 is detachably connected to the stopper 24 by a suitable method, e.g., by a thread 28.

FIG. 1 thus shows a generally known injection device 10 designed as a disposable syringe, which is prefilled and, after being removed from a blister package (not shown) and after removal of the safety cap 20, can be used in a known way for injection of the injectable product in the needle-equipped interior space 22.

FIG. 2 shows schematically the use of the injection device 10 in a needle-free injector 30. The needle-free injector 30 comprises a housing 32 as well as a clamping and operating device (not illustrated in detail), the functions of which will be discussed in greater detail below. A casing 34, which forms an interior space 36 to hold the injection device 10, is arranged inside the housing 32. The interior space 36 corresponds in its dimensions to the geometry of the supply container 12 with the cannula 18 and the attached safety cap 20. The ring bulge 40 on the supply container 12 is supported on a ring shoulder 42 of the casing 34. The supply container 12 with the safety cap 20 is thus surrounded by the casing 34 in a form-fitting manner.

A spring element 44 is also arranged inside the housing 32, supported at one end on the ring shoulder 42 of the casing 34 and supported on the housing 32 at the other end. FIG. 2 shows the spring element 44 in its stretched position. By means of a clamping device (not shown), the casing 34 is prestressed and locked here against the force of the spring element 44. By means of the release device (not shown), the spring element 44 can be released from its clamped locking position.

For use of the injection device 10 in the needle-free injector 30, the plunger rod 26 (FIG. 1) has been replaced by a plunger (NFI plunger) for needle-free injection, labeled as 46 on the whole. The plunger 46 comprises a guide element 48, which corresponds in its outside dimensions essentially to the plunger rod 26 and which may be inserted into the stopper 24 by means of the thread 28. The plunger rod 26 is first removed from the thread 28 by an appropriate rotational movement and replaced by the plunger 46. A core 50 within which there is a hollow needle 52 is provided within the guide element 48. The hollow needle 52 extends over the entire length of the plunger 46 and has a ground joint 54 on its end facing the stopper 24. After insertion of the plunger 46 into the stopper 24 and insertion of the injection device 10 into the needle-free injector 30, the ground joint 54 punctures the stopper 24 without leaving a residue by partial displacement of the hollow needle 52 toward the plunger 46, thereby establishing a fluid connection between the interior space 22 of the supply container 12 and a nozzle arrangement 56 of the needle-free injector 30 in a manner to be explained below.

It is clear on the basis of the schematic sectional view of the nozzle arrangement 56 shown in FIG. 3 that the hollow needle 52 extends up to an outlet nozzle 58. The outlet nozzle 58 constitutes the interface for needle-free injection into the tissue.

The nozzle arrangement 56 has an interior space 60, which is bordered by a stopper 62. The hollow needle 52 passes through the interior space 60 and has at least one outlet opening 64 in the area of the interior space 60. The stopper 62 likewise extends around the hollow needle 52 and is in contact with a plunger 66. Both the stopper 62 and the plunger 66 are displaceably guided with a seal on the hollow needle 52. The plunger 66 forms a ring bulge 68. This ring bulge 68 is guided with respect to a support 70 (FIG. 2), which is in turn displaceable against the force of a spring element 72. The spring element 72 is supported on the housing 32 of the needle-free injector 30, where it is fixedly mounted on the housing.

The needle-free injector 30 has the following function:

The injection device 10, still designed as a disposable syringe, is removed from the blister package. The plunger rod 26 is removed from the stopper 24 and replaced by the plunger 46. On insertion of this injection device, preconfigured in this way, into the housing 32, in particular into the prestressed casing 34, the hollow needle 52 is displaced in the direction of the stopper 24, so that the latter is punctured, establishing a fluid connection to the interior space 22. The cannula 18 and the safety cap 20 of the injection device 10 remain unaffected here and are introduced into the casing 34 as shown in FIG. 2.

The needle-free injector 30 is then placed with its nozzle arrangement 56 tightly against the skin. By releasing the spring element 44, the casing 34 is displaced within the housing 36 in the direction of the nozzle arrangement 56. There is a relative displacement here of the supply container 12 in relation to the stopper 24, which remains secured in its position by the plunger 46. The interior space 22 is thus reduced in size, thereby displacing the product, which is held in the interior space 22 and is to be injected. The product then passes through the hollow needle 52 and at least one outlet opening 64 first into the interior space 60 of the nozzle arrangement 56. In this way, the stopper 62 and the plunger 66 are displaced, so that a certain amount of product to be injected enters the interior space 60. Appropriate measures may be taken to be sure that any air in the interior space 60 and/or in the hollow needle 52 is not also injected. This may be, for example, an element that automatically absorbs air and which is integrated into the nozzle body. After a distance that is predetermined by the design, the ring bulge 68 comes in contact with the support 70, so that the prestressed spring element 72 is released abruptly. The plunger 66 and the stopper 62 are therefore displaced according to the direction of the arrow 74, which is indicated in FIG. 3. The product that is to be injected and in the meantime is in the interior space 60 is displaced back into the hollow needle 52 through at least one outlet opening 64. This results in a superimposed displacement of the product in the interior space 22 by the product in the interior space 60, the latter being discharged at the outlet nozzle 58 and injected into the tissue. This superimposed displacement leads to a pressure peak, so that a high-pressure stream with a high pressure and a small volume is created to form an injection channel in the tissue. After the product in the interior space 60 has been displaced, there is a buildup of pressure up to the injection pressure, which is established due to the parallel and persistent displacement of the product in the interior space 22. The product can thus be injected into the tissue through the injection channel created immediately previously. Thus, first a small volume of the product is injected at a high pressure and then a large volume of product is injected at a low pressure. Reference is made here to the content of WO 03/105934 A1 with regard to the advantages of creating an injection channel by means of a high pressure and small volume of product and subsequent introduction of the product to be injected with a larger volume and lower pressure in comparison with the volume and pressure during creation of the injection channel. The disclosure content is herewith included in the present description to this extent.

FIG. 4 shows a diagram of the basic curve of pressure P over time t and over distance s. Time t relates here to the release moment of the spring element 44 at time t₀, the subsequent filling of the interior space 60 up to time t₁, the pressure peak, which then builds up until time t₂ and the actual injection subsequently up to time t₃ (the interior space 22 is empty). Similarly, distance s refers to the spring deflection of the spring element 44. The spring element 44 is released at the distance s₀; the interior space 60 is filled at the spring deflection s₁; the high-pressure pulse occurs at spring deflection s₂ and the interior space 22 has been emptied and the spring element 44 has relaxed at spring deflection s₃.

It is clear that the curve of pressure P over time t and over distance s can be adjusted easily through the choice of the spring characteristics of the spring element 44 and of the spring element 72.

On the whole, this creates a ready-to-use needle-free injector 30 by using a conventional commercial injection device 10 (disposable syringe) and a plunger 46 with a few reliable manipulations that can be repeated without error for a needle-free injection. It is possible to decide just before administration of the product to be injected whether the injection is to be done through the cannula 18 with a needle or through the needle-free injector 30 without a needle.

According to an embodiment variant that is not shown here, it is possible to provide for the disposable syringe—i.e., the injection device 10—to comprise the NFI plunger 46. The plunger 46 would then be usable either as a plunger rod 26 for a needle-equipped injection or for needle-free injection by means of the injector 30.

### LIST OF REFERENCE NUMERALS

10 injection device
12 supply container
14 first outlet opening
16 operating device
18 cannula
20 safety cap
22 interior space (of the supply container 12)
24 stopper (of the interior space 22)
26 plunger rod
28 thread
30 needle-free injector
32 housing
34 casing
36 interior space (of the casing 34)
40 ring bulge (of the supply container 12)
42 ring shoulder
44 spring element
46 plunger
48 guide element
50 core
52 hollow needle
54 ground joint
56 nozzle arrangement
58 outlet nozzle
60 interior space (of the nozzle arrangement 56)
62 stopper (of the interior space 60)
64 outlet opening
66 plunger
68 ring bulge (of plunger 66)
70 support
72 spring element
P pressure
t time
s deflection

1. An injection device, in particular for a system for injection of an injectable product, comprising:
a supply container that holds the product,
a first outlet arrangement that uses a needle for transferring the product out of the supply container to the injection site,
an operating device for applying a pressure to the product in the supply container, such that the operating device comprises a second needle-free outlet opening for transferring the product from the supply container to the injection site or is arranged to be replaceable by the second needle-free outlet arrangement.

2. The injection device according to claim 1, wherein the needle-free outlet arrangement has a plunger comprising a nozzle arrangement for needle-free injection and a fluid connection between the supply container and the nozzle arrangement.

3. The injection device according to claim 1, wherein the nozzle arrangement comprises an interior space having a fluid connection to the hollow needle by means of at least one outlet opening.

4. The injection device according to claim 1, wherein the interior space is sealed by a stopper that is guided on the hollow needle with a seal and is displaceable relative thereto.

5. The injection device according to claim 1, wherein the stopper is displaceable into the interior space by the force of a spring element (72).

6. The injection device according to claim 1, wherein the injection device can be introduced into a needle-free injector.

7. The injection device according to claim 1, wherein the injector comprises a casing arranged inside a housing, so that the injection device can be displaced into the casing.

8. The injection device according to claim 1, wherein the casing holds the supply container and the needle-equipped outlet arrangement in a form-fitting manner.

9. The injection device according to claim 1, wherein the casing is displaceable due to the force of a spring element, entraining the supply container in the direction of the nozzle arrangement.

10. The injection device according to claim 1, wherein the hollow needle of the plunger punctures through a stopper that serves to seal the interior space of the supply container.

11. The injection device according to claim 1, wherein a guide element on the plunger, the hollow needle and the stopper are arranged so they are stationary in displacement of the casing toward the housing of the needle-free injector.
